(19) United States
(12) Patent Application Publication
(10) Pub. No.: US 2016/0103974 A1
(43) Pub. Date: Apr. 14, 2016

(54) PERSONALIZED ANTIBIOTIC DOSING PLATFORM

(71) Applicant: LuminaCare Solutions Inc., Woburn, MA (US)

(72) Inventor: David Howe, Woburn, MA (US)

(21) Appl. No.: 14/877,194

(22) Filed: Oct. 7, 2015

Related U.S. Application Data
(60) Provisional application No. 62/061,727, filed on Oct. 9, 2014.

(51) Int. Cl. G06F 19/00; A61B 5/00

(52) U.S. Cl. CPC G06F 19/3456; G06F 19/3437; A61B 5/4848

(57) ABSTRACT

A personalized antibiotic dosing platform may comprise method and systems configured for: receiving infection data, wherein the infection data comprises a bacterial strain and a first bacterial load of the bacterial strain; receiving patient characteristics; receiving a prescribed drug and a prescribed dosage; receiving historic bacterial response data; receiving at least one pharmacokinetic model; applying at least one algorithm based on at least one of the following: the at least one pharmacokinetic model, and the historic bacterial response data, to compute a time interval for receiving a measurement of a second bacterial load; providing the computed time interval to a user; receiving the second bacterial load after an actual time interval; analyzing data based on at least two of the following: the first bacterial load, the second bacterial load, the actual time interval, the prescription drug, and the prescription dosage; and providing a treatment recommendation.

### RELATED APPLICATION

Under provisions of 35 U.S.C. §119(e), the Applicant claims the benefit of U.S. provisional application No. 62/061,727, filed Oct. 9, 2014, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### FIELD OF DISCLOSURE

The present disclosure relates to the process of using predictive algorithms in determining whether bacteria responsible for a bacterial infection in a patient are sensitive to an administered antibiotic.

### BACKGROUND

The rates of resistant bacterial infections are increasing in the United States and throughout the world. Each year at least 2 million people in the United States acquire resistant bacterial infections with at least 23,000 people dying as a result of the antibiotic resistant bacterial infection. Antibiotic-resistant bacterial infections add $10-$20 billion in excess costs to the U.S. healthcare system. In general, patients with resistant bacterial infections have longer hospital stays, prolonged treatments, have more physician and hospital visits and have greater disability and mortality than patients with drug susceptible bacterial infections.

Giving the appropriate antibiotic in a timely manner has been shown to decrease the rates of mortality and length of hospital stay for patients with resistant-bacterial infections. Current methods using bacterial culture methods can take 2 to 3 days or more to be able to identify which drugs are effective against the pathogen. FIG. 1 illustrates the current methods for identifying effective drugs. Several methods are currently being used or developed to decrease the time to bacterial strain identification including next-generation sequencing, quantitative PCR, fluorescent assays, and mass spectrometry. These methods can identify resistance mechanisms but cannot reliably determine the susceptibility of the pathogen to potential antibiotics.

There is a significant need in the art for improvements in the time for identifying if a patient has a resistant bacterial infection. Specifically, the prior art is deficient in guiding personalized treatment for patients in the hospital setting for selecting drugs for the rapid treatment of drug-resistant bacterial infections. Further, the prior art is deficient in providing a biomarker for the efficacy of the treatment.

Citation: Revilla N, Martín-Suárez A, Pérez M P, González F M, Fernández de Gatta M. Vancomycin dosing assessment in intensive care unit patients based on a population pharmacokinetic/pharmacodynamics simulation. Br. J. Clin. Pharmacol. 2010; 70: 201-212.

### Brief Overview

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

A personalized antibiotic dosing platform may comprise method and systems configured for: receiving infection data, wherein the infection data comprises a bacterial strain and a first bacterial load of the bacterial strain; receiving patient characteristics; receiving a prescribed drug and a prescribed dosage; receiving historic bacterial response data; receiving at least one pharmacokinetic model; applying at least one algorithm based on at least one of the following: the at least one pharmacokinetic model, and the historic bacterial response data, to compute a time interval for receiving a measurement of a second bacterial load; providing the computed time interval to a user; receiving the second bacterial load after an actual time interval; analyzing data based on at least two of the following: the first bacterial load, the second bacterial load, the actual time interval, the prescription drug, and the prescription dosage; and providing a treatment recommendation.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 shows current treatment paradigm for patients with suspected bacterial infection;

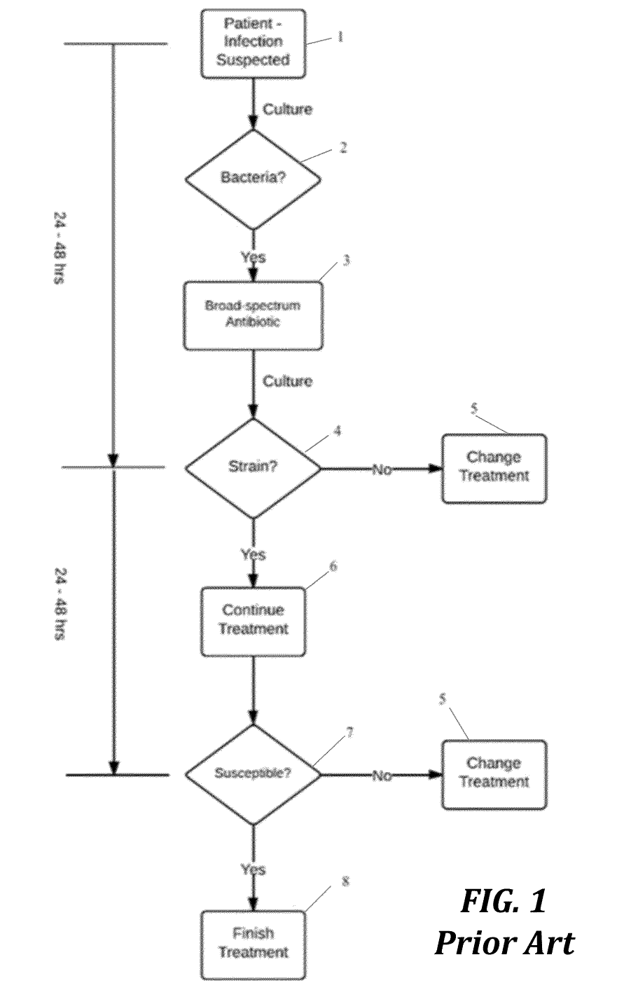

FIG. 2 illustrates one possible operating environment through which a platform consistent with embodiments of the present disclosure may be provided;

FIG. 3 is a flow chart of a method for providing a personalized bacterial dosing software platform;

FIG. 4 is a block diagram of a system including a computing device for performing the method of FIG. 3;

FIG. 5 is a chart describing time kill kinetics of a single clinical isolate of bacteria with various concentrations of antibiotics over time;

FIG. 6 is a chart describing the distribution of bacteria drug responses as a function of degree of resistance of isolates of the strain of bacteria against an antibiotic including the frequency of the number of isolates as a function of the isolates minimum inhibitory concentration (MIC); and

FIG. 7 is a chart describing the variation of exposure of typical drug in population of patients at a dose of 100 mg per kg body weight.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the contexts referred to below, embodiments of the present disclosure are not limited to use only in this context.

### I. DEFINITIONS

Throughout this disclosure, the terms used are defined by the examples that follow. It should be noted that other the definitions may be suitable for these terms and the definitions provided below are non-limiting examples of suitable definitions.

Bioanalysis, bioanalytical method or bioanalytical testing may mean any analytical technique or process known in the art to determine the amount or concentration of a therapeutic agent or metabolite of a therapeutic agent in a patient sample. Techniques include, but are not limited to, high-performance liquid chromatography, mass spectrometry, LC-MS, gas chromatography, GC-MS, radioimmunoassay, enzyme linked immunosorbent assay, and other techniques for quantitating therapeutic levels in biological samples known in the art.

Diagnostic testing may mean any analytical technique or process known in the art to determine bacterial strain identification and determine the amount or concentration of bacteria in a patient sample. Techniques include culture plating, quantitative polymerase chain reaction, fluorescence, imaging and other techniques that can be used for quantitating bacteria in biological samples known in the art.

A biological sample may be any material obtained from a patient, which contain the therapeutic agent or corresponding metabolites. Some examples include blood, plasma, urine, feces, tissue samples, tumor, and biopsy tissues.

Physician may be understood to include any professional licensed or trained to treat or take patient data and/or patient biological samples. The list includes physicians, doctors, clinicians, health care workers, nurses, technicians and others.

Dosage may apply to the size, frequency, administration route, formulation, co-medications, and number of doses of at least one therapeutic agent.

A patient may be a human or other mammal suffering from a disease, in need of treatment for a disease, or in need of testing of screening for a disease.

Patient data may include, but is not limited to, age, gender, weight, height, allergies, renal function, impaired metabolic function, drug concentrations from therapeutic drug monitoring, previous diseases, other therapies/medications, diet, physical condition, disease states, family history, disease progression, genetic information related to disease progression or distribution of therapeutic agent, and other relevant information.

Population pharmacokinetic models or a pharmacokinetic model may predict individual therapeutic agent concentrations in blood as a function of time for an administered dose of therapeutic agent.

### II. BACTERIAL RESPONSE OVERVIEW

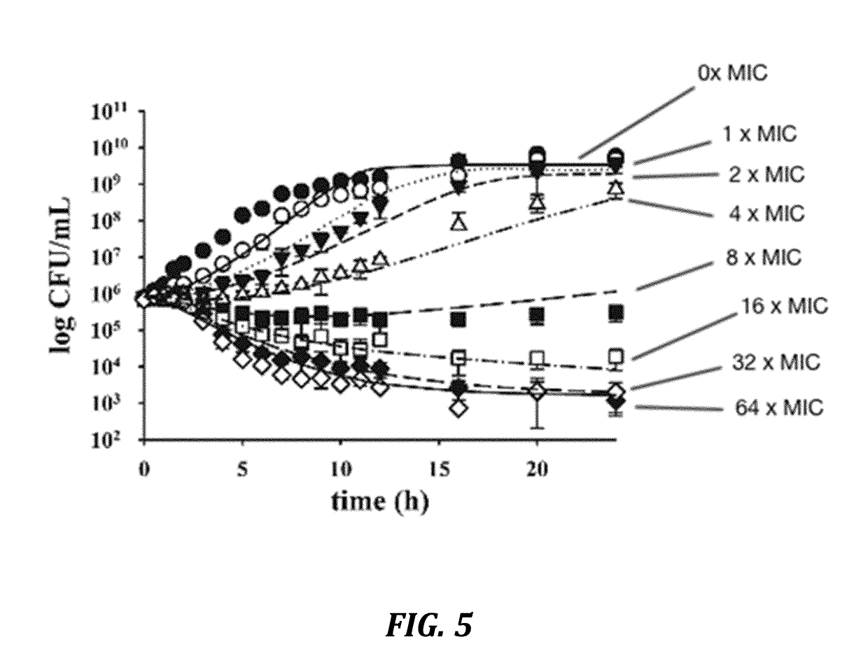

An overview of the concept for the bacterial response to an antibiotic drug module of the disclosure is depicted in FIG. 5. The killing effects of an antibiotic as depicted in FIG. 5 are generally described using the following base equation with different sub-populations of a clinical isolate of bacteria with differing susceptibilities to an antibiotic.

B=B₁+B₂+B₃+ . . . +Bₙ Eq. (I)

Equation (I) describes the population of bacteria, B, made up of sub-populations of bacteria B₁ to Bₙ that have different susceptibilities to an antibiotic.

$$\begin{matrix}\frac{d {B}_{1}}{d t} = {k}_{{growthB}_{1}} \times {B}_{1} - {k}_{death} \times {B}_{1} - \frac{{E}_{{maxB}_{1}} \times {C}^{γ}}{{C}^{γ} + {EC}_{50 {B}_{1}}^{γ}} \times {B}_{1} \\ \frac{d {B}_{2}}{d t} = {k}_{{growthB}_{2}} \times {B}_{2} - {k}_{death} \times {B}_{2} - \frac{{E}_{{maxB}_{2}} \times {C}^{γ}}{{C}^{γ} + {EC}_{50 {B}_{2}}^{γ}} \times {B}_{2} \\ ⋮ \\ \frac{d {B}_{n}}{d t} = {k}_{{growthB}_{n}} \times {B}_{n} - {k}_{death} \times {B}_{n} - \frac{{E}_{{maxB}_{n}} \times {C}^{γ}}{{C}^{γ} + {EC}_{50 n}^{γ}} \times {B}_{n}\end{matrix} \qquad Eq . ( II )$$

Equation (II) describes the change in bacterial subpopulations as a function of time in the presence of a single antibiotic. The parameters of Equation (II) include C for the concentration of antibiotic present, k_{growthBn} for the growth rate of bacterial subpopulations. The natural death rate of bacteria is k_{death}. The maximum killing rate of the antibiotic for the bacterial subpopulation is E_{maxBn}. The effective concentration of antibiotic for half-maximal killing rate is EC_{50Bn} and γ is the Hill coefficient.

The parameters of Equation (II) vary for each clinical isolate of bacteria. The overall responses of the population of a strain of bacteria with different degrees of response to an antibiotic can be described in Equation (III).

$$\begin{matrix}{EC}_{50 {B}_{1}} = {θ}_{{EC}_{50} {B}_{1}} \times exp ( {η}_{{EC}_{50} {B}_{1}} ) \\ {EC}_{50 {B}_{2}} = {θ}_{{EC}_{50} {B}_{2}} \times exp ( {η}_{{EC}_{50} {B}_{2}} ) \\ ⋮ \\ {EC}_{50 {B}_{n}} = {θ}_{{EC}_{50} {B}_{n}} \times exp ( {η}_{{EC}_{50} {B}_{n}} ) \\ {k}_{{growthB}_{1}} = {θ}_{{k}_{{growthB}_{1}}} \times exp ( {η}_{{k}_{{growthB}_{1}}} ) \\ {k}_{{growthB}_{2}} = {θ}_{{k}_{{growthB}_{2}}} \times exp ( {η}_{{k}_{{growthB}_{2}}} ) \\ ⋮ \\ {k}_{{growthB}_{n}} = {θ}_{{k}_{{growthB}_{n}}} \times exp ( {η}_{{k}_{{growthB}_{n}}} ) \\ {E}_{max} = {θ}_{{E}_{max}} + {η}_{{E}_{max}} \\ γ = {θ}_{γ} + {η}_{γ}\end{matrix} \qquad Eq . ( III )$$

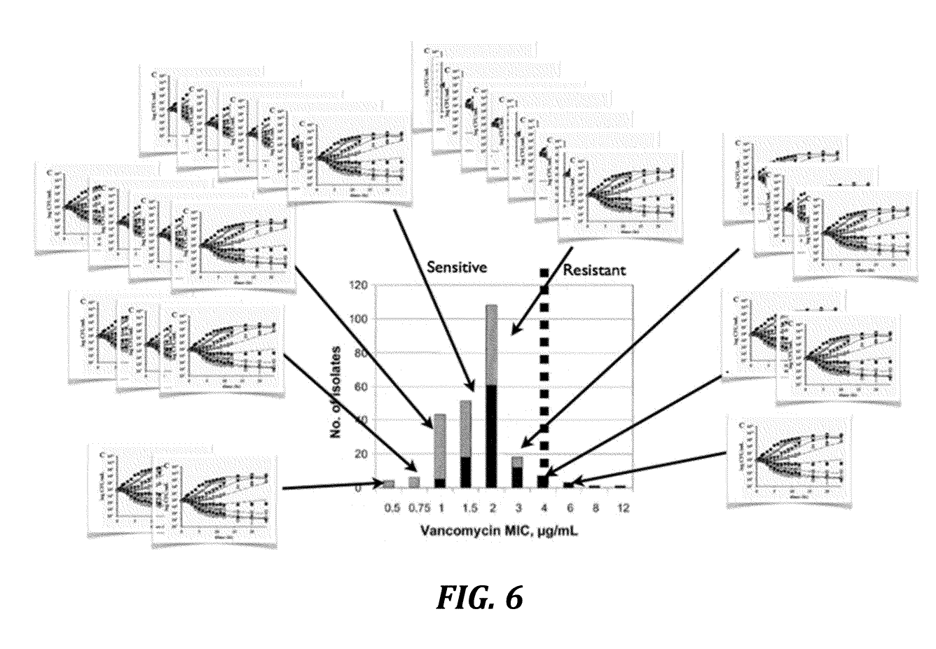

In Equation (III), θ is the population mean of the corresponding parameter and η is the deviation from the mean of each bacterial clinical isolate with zero mean and variance ω². An overview of the concept for the distribution of bacterial responses of clinical isolates of a strain of bacteria to an antibiotic drug module of the disclosure is depicted in FIG. 6. There is a distribution of values of the parameters given in Equation (III), θ, and the distribution of starting concentration of different bacterial sub-populations based on the degree of resistance of the clinical isolate to the drug as determined by the isolates' minimum inhibitory concentration. The probability of each value is determined through Bayesian analysis or other similar techniques from a collection of time-kill experiments as depicted in FIG. 5 for various isolates of a strain of bacteria with different degrees of antibiotic resistance.

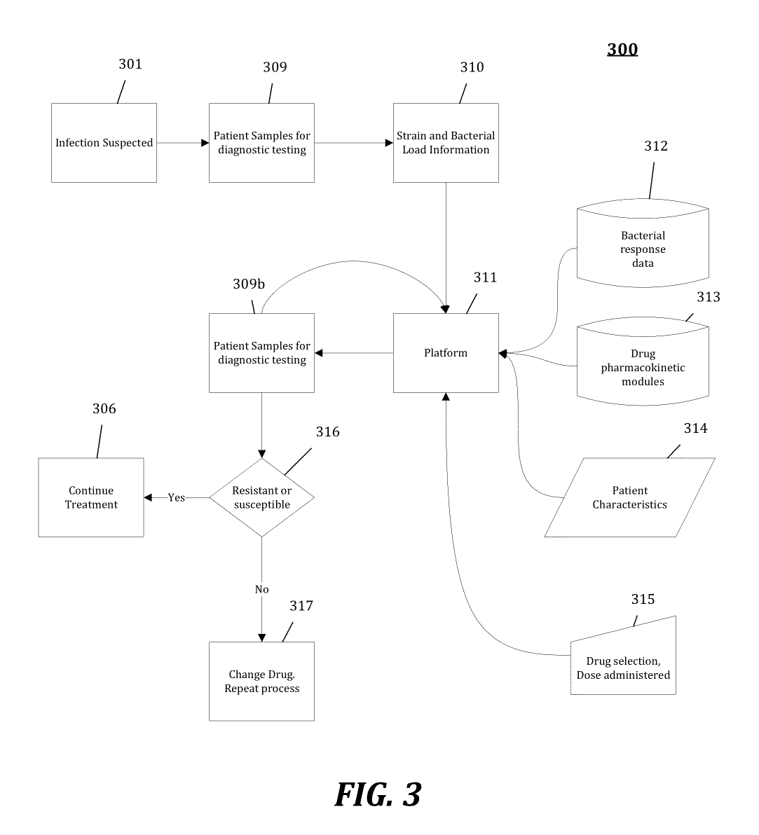

The current invention determines the bacterial response in a patient as depicted in FIG. 3. The physician suspects that the patient may have an infection. Patient samples are collected for rapid diagnostic tests. The results of the tests are entered into the software platform and include bacterial strain and bacterial load or the number of bacteria in the patient sample. The physician selects the drug, amount administered and the route of administration for the bacterial strain and includes patient characteristics for determining the total amount of drug dosed. These characteristics are dependent on the drug selected. The drug exposure of the patient is simulated based on prior art techniques and population pharmacokinetic equations as exemplified in Equations (IV-VII) for vancomycin. Vancomycin is used as an example and this approach can be widely applied.

The platform calculates another time for patient sample to be collected after initial dose of the chosen antibiotic. Monte Carlo simulations and other modifications of Monte Carlo simulations are used to determine the sampling point to ensure that resistant and sensitive bacterial responses can be predicted with 95% confidence.

A second sample is thus obtained for diagnostic testing. The bacterial load is determined at this second time point. Depending on the second bacterial load value obtained, the isolate is either resistant to the antibiotic of sensitive. If resistant, the antibiotic needs to be changed. If the bacterial isolate is sensitive, the parameters describing the clinical isolate are calculated using Bayesian methods and the bacterial responses can be modeled for the course of treatment in the platform informing the physician how long to treat the patient. If resistant, the drug can be altered and the process repeated.

### III. PLATFORM CONFIGURATION

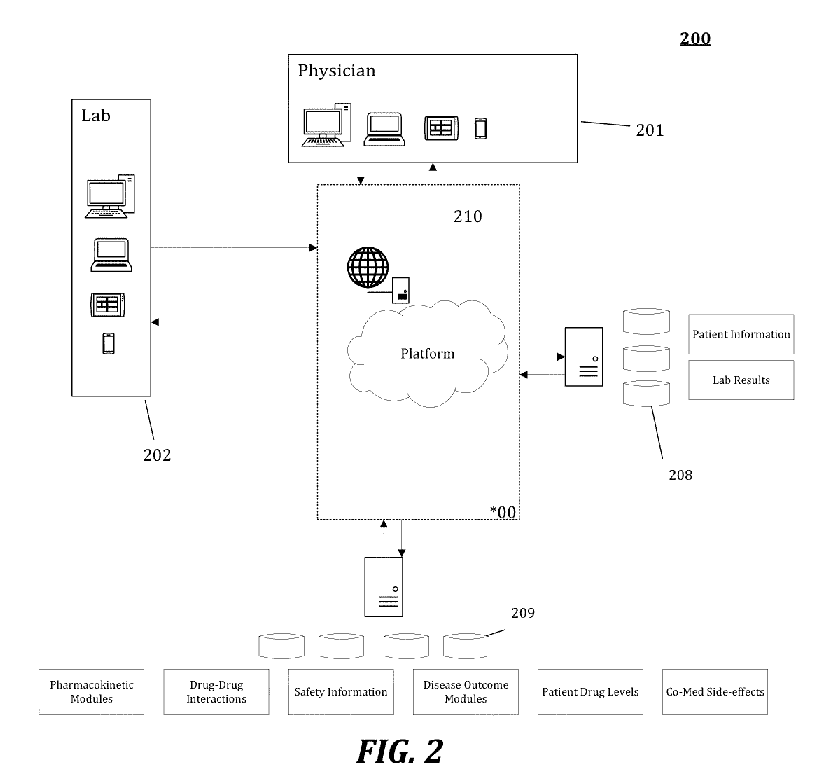

FIG. 2 illustrates one possible operating environment through which a platform consistent with embodiments of the present disclosure may be provided. By way of non-limiting example, a personalized antibiotic dosing platform 200 may be hosted on a centralized server 210, such as, for example, a cloud computing service. A users may access platform 200 through a software application. The software application may be embodied as, for example, but not be limited to, a website, a web application, a desktop application, and a mobile application compatible with a computing device 400. One possible embodiment of the software application may be provided by LuminaCare Solutions Inc.

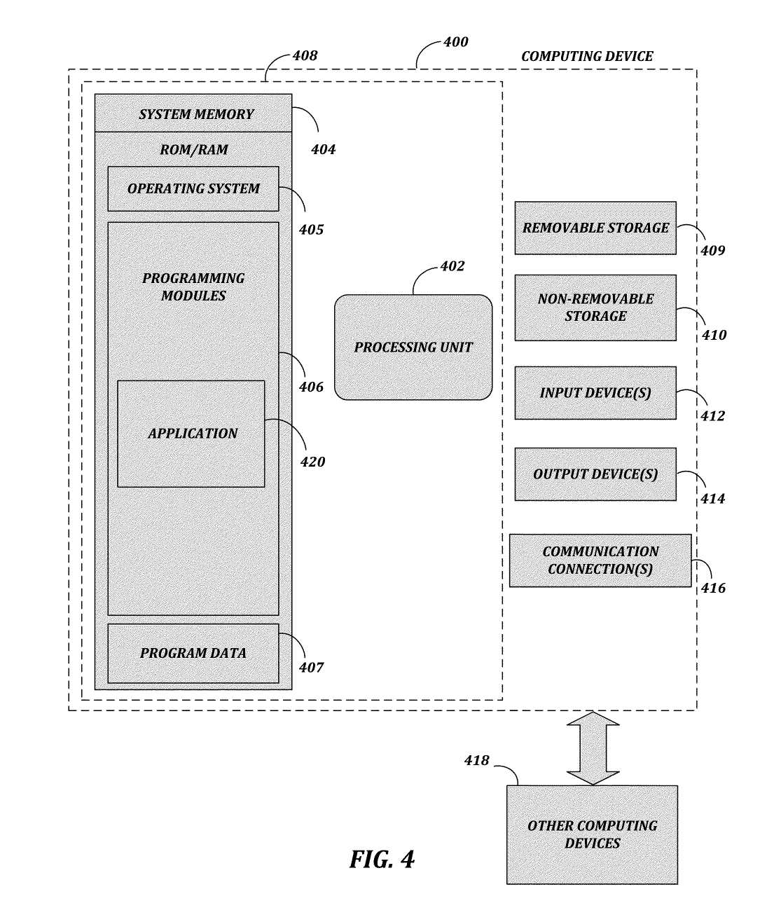

As will be detailed with reference to FIG. 4 below, the computing device through which the platform may be accessed may comprise, but not be limited to, for example, a desktop computer, laptop, a tablet, or mobile telecommunications device. As will be detailed below, these devices may be used by physicians and laboratories. Although the present disclosure is written with reference to particular computing devices, it should be understood that any computing device may be employed to provide the various embodiments disclosed herein.

Embodiments consistent with the present disclosure may be comprised of a cloud-based software system that have interfaces 201 for the physician or caregiver to enter information about the patient including site of infection, patient characteristics such as age, for example, weight, height, renal capacity and other characteristics necessary for appropriate treatment of initial broad-spectrum antibiotic. The diagnostic tests may be entered through interfaces 202 with the cloud-based software by hospital lab or other outsourced lab.

The information provided to platform 200 includes the bacterial pathogen and the amount of bacteria present at the site of infection. The antibiotic(s) and dosage information provided to platform 200 may be based on the bacterial pathogen, site of infection and severity of the infection. The expected drug levels are calculated as exemplified for vancomycin in population pharmacokinetics observed in patients in the intensive care unit. The method is not limited to its application to vancomycin, but is only used as an example. The base model describing the concentrations of vancomycin at time t in the plasma after dosage is calculated as follows:

$$\frac{d C}{d t} = - \frac{CL}{V} \times C \qquad Eq . ( IV )$$

Equation (IV) describes the rate of change of vancomycin over time. The parameters and constants of Equation (IV) include C for the concentration of vancomycin at time t, CL for vancomycin plasma clearance and V for the volume of distribution of vancomycin.

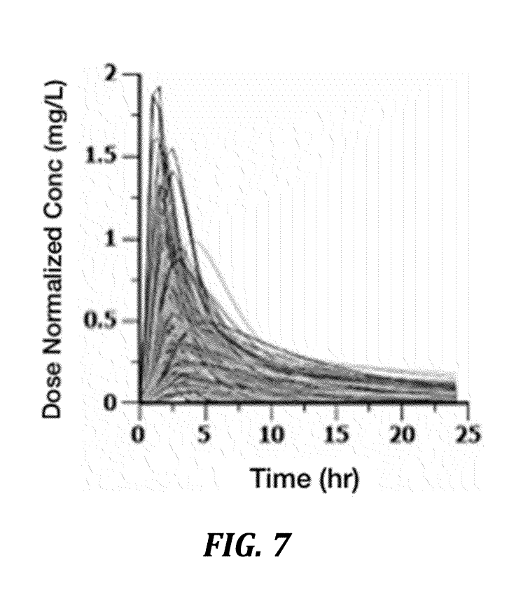

The typical range of vancomycin exposures observed in the general population of 1000 patients in displayed in FIG. 7. The exposure profile is personalized to the patient through matching the patient characteristics with the appropriate population pharmacokinetic model contained in the drug database.

CLᵢⱼ=CL×e^{ηCL} Eq. (V)

Equation (V) describes the clearance, CLᵢⱼ, for the i^{th} subject, CL is the mean clearance of vancomycin in the population and ηCL is a random inter-individual variable that is normally distributed with zero mean and variance ω.

Vᵢⱼ=V×e^{ηV} Eq. (VI)

Equation (VI) describes the volume of distribution, Vᵢⱼ, for the i^{th} subject, V is the mean volume of distribution of vancomycin in the population and ηV is a random inter-individual variable that is normally distributed with zero mean and variance ω.

CL=θ₁×CL_{cr}+AGE^{θ}^{₂} Eq. (VII)

Equation (VII) describes the mean clearance of vancomycin as a function of patient age and renal sufficiency as measured by creatinine clearance. The population constants θ₁ and θ₂ are 0.67 and −0.24 for adult patients in intensive care units.

Based on the information provided by the physician to platform 200 including lab results 208, dose and patient information, the platform 200 predicts drug expose over time using databases and models 209. A second sampling time is calculated to differentiate between a sensitive versus a resistant bacterial response. The quantity of bacteria is determined for the patient at the second time point. The platform 200 informs physician 201 if infection is resistant to the antibiotic. If resistant, physician can select next line medication from platform 200 and the platform 200 calculates another sampling time point to distinguish whether the infection is sensitive or resistant to new treatment. For a sensitive infection, the platform 200 integrates pharmacokinetic models 209 with bacterial database information using Bayesian analysis, Monte Carlo and modified Markov Monte Carlo simulation and the 1^{st} and 2^{nd} bacterial concentrations determined for the patient. The physician 201 may modify dosing amounts, dosing frequency and length of treatment. In turn, the platform 200 may predict the bacterial concentrations over time to determine length of treatment.

### IV. PLATFORM OPERATION

FIG. 3 is a flow chart setting forth the general stages involved in a method 300 consistent with an embodiment of the disclosure for providing platform 200. Method 300 may be implemented using a computing device 400 as described in more detail below with respect to FIG. 4.

Although method 300 has been described to be performed by platform 200, it should be understood that computing device 400 may be used to perform the various stages of method 300. Furthermore, in some embodiments, different operations may be performed by different networked elements in operative communication with computing device 400. For example, server 210 may be employed in the performance of some or all of the stages in method 300. Moreover, server 210 may be configured much like computing device 400.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 300 will be described in greater detail below.

Method 300 may begin at starting block 301 where an infection is suspected and proceed to stage 309, where biological samples are removed and tested as described in prior art.

From stage 309, where biological samples are removed and tested as described in prior art, method 300 may advance to stage 310 where the strain and bacterial load at the site of infection are identified.

From stage 310, method 300 may continue to stage 311 where the bacterial strain, bacterial load and patient characteristics are entered into platform 200 (e.g., via interfaces 201 and/or 202). In addition, computing device 400 may receive the selected drug and dose as prescribed by a physician 315. Further, computing device 400 may receive algorithms derived from historic bacterial response data 312, drug human pharmacokinetic models 313 and patient characteristics 314 to derive a time interval for another collection of a biological sample from the patient after administration of selected drug at the selected dose.

From stage 311, where relevant information is input into the platform, doses are administered and biological samples are removed and tested with quantitative diagnostic tests as described in prior art in stage 309b. The results of the diagnostic tests and time elapsed are then input into computing device 400. The drug exposure of the patient is predicted based on population pharmacokinetic equations as exemplified in Equations (IV-VII) for vancomycin. The platform calculates another time for patient sample to be collected after initial dose of the chosen antibiotic. Monte Carlo simulations and other modifications of Monte Carlo simulations may be used to determine the sampling point to ensure that resistant and sensitive bacterial responses can be predicted with 95% confidence.

After computing device 400 receives the bacterial strain, quantity of bacteria and patient characteristics in stage 309b, method 300 may proceed to stage 316 where the results are analyzed according to the described method and acted upon. For example, a physician may determine that the results are on track for a timely recovery. Alternatively, a physician may determine that the results are inadequate and the drug or dosage must be altered.

Once the results are acted upon in stage 316, method 300 may then end at stage 306 by continuing the current treatment or end at 317 by changing the drug and repeating method 300.

### V. PLATFORM ARCHITECTURE

Platform 200 may be embodied as, for example, but not be limited to, a website, a web application, a desktop application, and a mobile application compatible with a computing device. The computing device may comprise, but not be limited to, a desktop computer, laptop, a tablet, or mobile telecommunications device. Moreover, platform 200 may be hosted on a centralized server, such as, for example, a cloud computing service. Although method 300 has been described to be performed by a computing device 400, it should be understood that, in some embodiments, different operations may be performed by different networked elements in operative communication with computing device 400.

FIG. 4 is a block diagram of a system including computing device 400. Consistent with an embodiment of the disclosure, the aforementioned memory storage and processing unit may be implemented in a computing device, such as computing device 400 of FIG. 4. Any suitable combination of hardware, software, or firmware may be used to implement the memory storage and processing unit. For example, the memory storage and processing unit may be implemented with computing device 400 or any of other computing devices 418, in combination with computing device 400. The aforementioned system, device, and processors are examples and other systems, devices, and processors may comprise the aforementioned memory storage and processing unit, consistent with embodiments of the disclosure.

With reference to FIG. 4, a system consistent with an embodiment of the disclosure may include a computing device, such as computing device 400. In a basic configuration, computing device 400 may include at least one processing unit 402 and a system memory 404. Depending on the configuration and type of computing device, system memory 404 may comprise, but is not limited to, volatile (e.g. random access memory (RAM)), nonvolatile (e.g. read-only memory (ROM)), flash memory, or any combination. System memory 404 may include operating system 405, one or more programming modules 406, and may include a program data 407. Operating system 405, for example, may be suitable for controlling computing device 400's operation. In one embodiment, programming modules 406 may include calculation and extrapolation programs. Furthermore, embodiments of the disclosure may be practiced in conjunction with a graphics library, other operating systems, or any other application program and is not limited to any particular application or system. This basic configuration is illustrated in FIG. 4 by those components within a dashed line 408.

Computing device 400 may have additional features or functionality. For example, computing device 400 may also include additional data storage devices (removable and/or non-removable) such as, for example, magnetic disks, optical disks, or tape. Such additional storage is illustrated in FIG. 4 by a removable storage 409 and a non-removable storage 410. Computer storage media may include volatile and nonvolatile, removable and non-removable media implemented in any method or technology for storage of information, such as computer readable instructions, data structures, program modules, or other data. System memory 404, removable storage 409, and non-removable storage 410 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 400. Computing device 400 may also have input device(s) 412 such as a keyboard, a mouse, a pen, a sound input device, a touch input device, etc. Output device(s) 414 such as a display, speakers, a printer, etc. may also be included. The aforementioned devices are examples and others may be used.

Computing device 400 may also contain a communication connection 416 that may allow device 400 to communicate with other computing devices 418, such as over a network in a distributed computing environment, for example, an intranet or the Internet. Communication connection 416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 404, including operating system 405. While executing on processing unit 402, programming modules 406 (e.g., computational application 420) may perform processes including, for example, one or more of method 200's stages as described above. The aforementioned process is an example, and processing unit 402 may perform other processes. Other programming modules that may be used in accordance with embodiments of the present disclosure may include electronic mail and contacts applications, word processing applications, spreadsheet applications, database applications, slide presentation applications, drawing or computer-aided application programs, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

### VI. CLAIMS

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A method comprising:
receiving infection data, wherein the infection data comprises a bacterial strain and a first bacterial load of the bacterial strain;
receiving patient characteristics;
receiving a prescribed drug and a prescribed dosage;
receiving historic bacterial response data;
receiving at least one pharmacokinetic model;
applying at least one algorithm based on at least one of the following: the at least one pharmacokinetic model, and the historic bacterial response data, to compute a time interval for receiving a measurement of a second bacterial load;
providing the computed time interval to a user;
receiving the second bacterial load after an actual time interval;
analyzing data based on at least two of the following: the first bacterial load, the second bacterial load, the actual time interval, the prescription drug, and the prescription dosage; and
providing a treatment recommendation.

2. The method of claim 1, wherein providing the treatment recommendation comprises providing at least one of the following: a change of the prescription drug recommendation, a change of the prescription dosage recommendation, a change of the prescription frequency recommendation, and an indication that the bacterial strain is resistant to the prescription.

3. The method of claim 1, wherein analyzing the data comprises predicting a bacterial load over time and predicting a length of treatment.

4. The method of claim 1, wherein the patient characteristics comprise at least one of the following: a patient's sex, the patient's weight, the patient's age, the patient's other prescriptions and doses.

5. The method of claim 1, wherein applying the at least one algorithm based on the at least one of the following: the at least one pharmacokinetic model, and the historic bacterial response data to compute the time interval for receiving the measurement of the second bacterial load comprises applying a Monte Carlo simulation.

6. The method of claim 1, wherein applying the at least one algorithm based on the at least one of the following: the at least one pharmacokinetic model, and the historic bacterial response data to compute the time interval for receiving the measurement of the second bacterial load comprises applying the at least one algorithm to determine the time interval to ensure that resistant and sensitive bacterial responses can be predicted with 95% confidence.

7. The method of claim 1, further comprising adding patient data to the historic data, wherein the patient data comprises: the bacterial strain, the first bacterial load, the prescription drug, the prescription dosage, the second bacterial load, and the actual time interval.

8. A computer-readable medium comprising a set of instructions, which when executed perform a method comprising:
receiving infection data, wherein the infection data comprises a bacterial strain and a first bacterial load of the bacterial strain;
receiving patient characteristics;
receiving a prescribed drug and a prescribed dosage;
receiving historic bacterial response data;
receiving at least one pharmacokinetic model;
applying at least one algorithm based on at least one of the following: the at least one pharmacokinetic model, and the historic bacterial response data, to compute a time interval for receiving a measurement of a second bacterial load;
providing the computed time interval to a user;
receiving the second bacterial load after an actual time interval;
analyzing data based on at least two of the following: the first bacterial load, the second bacterial load, the actual time interval, the prescription drug, and the prescription dosage; and
providing a treatment recommendation.

9. The computer readable medium of claim 8, wherein providing the treatment recommendation comprises providing at least one of the following: a change of the prescription drug recommendation, a change of the prescription dosage recommendation, a change of the prescription frequency recommendation, and an indication that the bacterial strain is resistant to the prescription.

10. The computer readable medium of claim 8, wherein analyzing the data comprises predicting a bacterial load over time and predicting a length of treatment.

11. The computer readable medium of claim 8, wherein the patient characteristics comprise at least one of the following: a patient's sex, the patient's weight, the patient's age, the patient's other prescriptions and doses.

12. The computer readable medium of claim 8, wherein applying the at least one algorithm based on the at least one of the following: the at least one pharmacokinetic model, and the historic bacterial response data to compute the time interval for receiving the measurement of the second bacterial load comprises applying a Monte Carlo simulation.

13. The computer readable medium of claim 8, wherein applying the at least one algorithm based on the at least one of the following: the at least one pharmacokinetic model, and the historic bacterial response data to compute the time interval for receiving the measurement of the second bacterial load comprises applying the at least one algorithm to determine the time interval to ensure that resistant and sensitive bacterial responses can be predicted with 95% confidence.

14. The computer readable medium of claim 8, further comprising adding patient data to the historic data, wherein the patient data comprises: the bacterial strain, the first bacterial load, the prescription drug, the prescription dosage, the second bacterial load, and the actual time interval.

15. A system comprising:
a memory storage; and
a processing unit coupled with the memory storage, wherein the processing unit is operative to:
receive infection data, wherein the infection data comprises a bacterial strain and a first bacterial load of the bacterial strain,
receive patient characteristics,
receive prescribed drug and a prescribed dosage,
receive historic bacterial response data,
receive at least one pharmacokinetic model,
apply at least one algorithm based on at least one of the following: the at least one pharmacokinetic model, and the historic bacterial response data, to compute a time interval for receiving a measurement of a second bacterial load,
provide the computed time interval to a user,
receive the second bacterial load after an actual time interval,
analyze data based on at least two of the following: the first bacterial load, the second bacterial load, the actual time interval, the prescription drug, and the prescription dosage, and
provide a treatment recommendation.

16. The system of claim 15, wherein the treatment recommendation comprises at least one of the following: a change of the prescription drug recommendation, a change of the prescription dosage recommendation, a change of the prescription frequency recommendation, and an indication that the bacterial strain is resistant to the prescription.

17. The system of claim 15, further operative to analyze the data by predicting at least one of the following: a bacterial load over time and a length of treatment.

18. The system of claim 15, wherein the at least one algorithm comprises a Monte Carlo simulation.

19. The system of claim 15, wherein the system is further operative to determine the time interval to ensure that resistant and sensitive bacterial responses can be predicted with 95% confidence.

20. The system of claim 15, wherein the processing unit is further operative to add patient data to the historic data, wherein the patient data comprises: the bacterial strain, the first bacterial load, the prescription drug, the prescription dosage, the second bacterial load, and the actual time interval.
